(19) United States
(12) Patent Application Publication
(10) Pub. No.: US 2024/0165001 A1
(43) Pub. Date: May 23, 2024

(54) Oral Compositions and Related Methods

(71) Applicant: Colgate-Palmolive Company, New York, NY (US)

(72) Inventors: Payal ARORA, Lebanon, NJ (US); Norbert HUBER, Basel (CH); Zhigang HAO, Bridgewater, NJ (US); Jeannine LOETSCHER, Ettingen (CH); Damien ROGALLE, Kembs (FR)

(21) Appl. No.: 18/511,130

(22) Filed: Nov. 16, 2023

Related U.S. Application Data
(60) Provisional application No. 63/425,864, filed on Nov. 16, 2022.

(51) Int. Cl. A61K 8/41; A61K 8/21; A61K 8/27; A61K 8/34; A61Q 11/00

(52) U.S. Cl. CPC A61K 8/41; A61K 8/21; A61K 8/27; A61K 8/347; A61Q 11/00

(57) ABSTRACT

The present disclosure relates to oral care compositions containing amines, fluoride ion sources, zinc-containing compounds and one or more stilbenoids (e.g., resveratrol), as well as related methods for use of said compositions.

### FIELD

The present disclosure relates to oral care compositions containing amines, fluoride ion sources, zinc-containing compounds and one or more stilbenoids (e.g., resveratrol), as well as related methods for use of said compositions.

### BACKGROUND

Oral hygiene compositions, by their cleaning action, make a contribution to the hygiene of the oral cavity and thus to the preservation of the health of teeth and gums. The cleaning action of these oral hygiene compositions is customarily supplemented by admixture of active compounds which prevent or control pathological symptoms in the oral cavity, in particular also the formation of bacterial films on the teeth (i.e., plaque).

These films consist of polysaccharides, primarily of dextrans. In addition to the low-molecular weight sugars, these polysaccharides form a source of nutrition for the plaque bacteria, which are mainly streptococci and lactobacillaceae. The plaque bacteria gradually break down the polysaccharides to form acidic degradation products (e.g., pyruvic acid, lactic acid, etc.). The pH decrease resulting therefrom brings about the degradation of the tooth enamel known as caries. This condition may lead to further complications, such as gingivitis and/or periodontitis.

It has therefore already been attempted to take steps against the formation of pathological symptoms in the oral cavity using various oral hygiene compositions (e.g., toothpastes, rinsing solutions or dental gels). Active compounds already known the prior art include N-octadeca-9-enylamine hydrofluoride (international non-proprietary name “dectaflur”) and N′-octadecyl-N′,N,N-tris(2-hydroxyethyl)-1,3-propanediamine dihydrofluoride (international non-proprietary name “olaflur”). On oral use of the hygiene composition, these active compounds form a thin hydrophobic film on the tooth enamel, the amine hydrofluoride groups coming into contact with the tooth enamel. Thus, on the one hand the tooth enamel becomes more resistant to acid attacks on account of the CaF₂ covering layer formed, on the other hand the long-chain hydrocarbon residues form a hydrophobic layer which prevents the formation of deposits and the attack of the acidic degradation products on the tooth enamel.

Zinc is also a known antimicrobial agent used in oral care compositions like toothpastes or mouthrinses. Zinc is a known essential mineral for human health, and has been reported to help strengthen dental enamel and to promote cell repair. Unfortunately, conventional toothpaste formulations often require high concentrations of zinc, e.g., 2% by weight or more, to achieve efficacy. At this concentration, the zinc imparts a notably astringent taste to the composition. There is thus a need for improved antibacterial toothpaste formulations that do not suffer from the drawbacks of conventional compositions.

Some compounds, such as stilbenoids, may provide a way to supplement the effects of zinc. For example, stilbenoids are being studied for their impact as anti-inflammatory agents and potential antioxidants. However, certain stilbenoids, such as resveratrol, are believed to be challenging for formulation purposes given that they can have poor solubility and limited bioavailability. See, Salehi B, Mishra AP, Nigam M, Sener B, Kilic M, Sharifi-Rad M, Fokou PVT, Martins N, Sharifi-Rad J. “Resveratrol: A Double-Edged Sword in Health Benefits.” Biomedicines. 2018 Sep 9;6(3):91. Moreover, formulating compositions with resveratrol is believed to be challenging given its potential sensitivity to pH and temperature. See, Zupanc̆ic̆ S̆, Lavric̆ Z, Kristl J. “Stability and solubility of trans-resveratrol are strongly influenced by pH and temperature.” Eur J Pharm Biopharm. 2015 Jun; 93:196-204.

Accordingly, in view of the drawbacks and disadvantages to using various antimicrobials, such as zinc, there is a need for oral care compositions with anti-bacterial and/or anti-inflammatory efficacy, but which are stable and also palatable and desirable for a user.

### BRIEF SUMMARY

Provided herein are methods of in situ synthesis of amine fluorides from amine bases, e.g., without the use of hydrofluoric acid, where the resulting compositions are stable and further comprise one or more stilbenoid (e.g., resveratrol). Related compositions (e.g., oral care compositions and/or personal care compositions) are also disclosed. In one aspect, the disclosure provides oral care compositions with a storage stable amount of one or more stilbenoids, e.g., resveratrol. In one aspect, the oral care compositions comprise a complex that forms from the dissolution of amine fluoride and resveratrol that potentially provides stability for resveratrol in toothpaste and mouthwash formulations. In this aspect, the complex is an amine fluoride-resveratrol complex.

Thus, in a first aspect, the present disclosure is directed to an oral care composition comprising:
an amine base,
a fluoride source,
a zinc source selected from zinc lactate and zinc citrate; and
a stilbenoid (e.g., resveratrol).

### BRIEF DESCRIPTION OF FIGURES

FIG. 1A depicts a graph showing anti-oxidant activity of fresh sample of Formulation 1, Formulation 2, Formulation 3, and positive control (0.1% vitamin E). FIG. 1B depicts a graph showing anti-oxidant activity of aged sample of Formulation 1, Formulation 2, Formulation 3, and positive control (0.1% vitamin E). The aged samples are stored for 3 months at 40° C. and 75% relative humidity (RH).

FIG. 2A depicts a graph showing anti-inflammation activity of fresh sample of Formulation 1 and Formulation 2. FIG. 2B depicts a graph showing anti-oxidant activity (measured by Trolox equivalent) of aged sample of Formulation 1 and Formulation 2. The aged samples are stored for 3 months at 40° C. and 75% relative humidity (RH).

FIG. 3A depicts a graph showing anti-oxidant activity of fresh sample of Formulation 4, Formulation 5, Formulation 6, and positive control (0.1% vitamin E). FIG. 3B depicts a graph showing anti-oxidant activity of aged sample of Formulation 4, Formulation 5, Formulation 6, and positive control (0.1% vitamin E). The aged samples are stored for 3 months at 40° C. and 75% relative humidity (RH).

FIG. 4A depicts a graph showing anti-inflammation activity of fresh sample of Formulation 4, Formulation 5, Formulation 6, and positive control (0.1% vitamin E). FIG. 4B depicts a graph showing anti-oxidant activity of aged sample of Formulation 4, Formulation 5, Formulation 6, and positive control (0.1% vitamin E) as well as controls ((i) no IL-1β treatment and (ii) untreated in the presence of IL-1β). The aged samples are stored for 3 months at 40° C. and 75% relative humidity (RH).

FIG. 5 depicts LC/MS data regarding amine ingredients from amine fluoride materials.

FIG. 6 depicts LC/MS data demonstrating possible amine-resveratrol complexes.

### DETAILED DESCRIPTION

As used herein, an “oral care composition” refers to a composition for which the intended use includes oral care, oral hygiene, and/or oral appearance, or for which the intended method of use comprises administration to the oral cavity, and refers to compositions that are palatable and safe for topical administration to the oral cavity, and for providing a benefit to the teeth and/or oral cavity. The term “oral care composition” thus specifically excludes compositions which are highly toxic, unpalatable, or otherwise unsuitable for administration to the oral cavity. In some embodiments, an oral care composition is not intentionally swallowed, but is rather retained in the oral cavity for a time sufficient to affect the intended utility. The oral care compositions as disclosed herein may also be used in nonhuman mammals such as companion animals (e.g., dogs and cats), as well as by humans. In some embodiments, the oral care compositions as disclosed herein are used by humans. Examples of such compositions include, but are not limited to, toothpaste or a dentifrice, a mouthwash or a mouth rinse, a topical oral gel, a denture cleanser, sprays, toothpaste powders, tablets, mousse, foam, chewing gums and the like.

As used herein, the term “dentifrice” means paste, gel, or liquid formulations unless otherwise specified. The dentifrice composition can be in any desired form such as deep striped, surface striped, multi-layered, having the gel surrounding the paste, or any combination thereof. Alternatively, the oral composition may be dual phase dispensed from a separated compartment dispenser.

As used herein, the term “amine base” may refer to a primary amine base, a secondary amine base or a tertiary amine base. “Primary amine base” refers to a compound containing at least one amine in which the nitrogen atom is directly bonded to one carbon of any hybridization, except for carbonyl group carbons. “Secondary amine base” refers to a compound containing at least one amine in which the nitrogen atom is directly bonded to two carbons of any hybridization, except for carbonyl group carbons. “Tertiary amine base” refers to a compound containing at least one amine in which the nitrogen atom is directly bonded to three carbons of any hybridization, except for carbonyl group carbons. “Amine base” may be used to refer to compounds containing a plurality of primary, secondary and/or tertiary amine groups (e.g., a tertiary polyamine). In particular, the term “amine base” excludes acid addition salts (e.g., hydrochloride salts and hydrofluoride salts), and thus refers to the free base form of the molecule. Hydrofluoride derivatives of amines are referred to herein as “amine fluorides.” In methods for the production or manufacture of a composition containing an amine fluoride, an amine base may be a precursor to form the amine fluoride.

As used herein, the term “in situ” is used to refer to the formation of a chemical product (e.g., amine fluoride) in the oral care composition. For example, the reaction may be a salination reaction carried out by mixing an amine with a fluoride source and an acid, thus creating an amine fluoride and a salt. In some embodiments, in situ excludes the possibility of formation of the reaction product in a first reaction vessel (for example, at a first location), and subsequent addition of the reaction product to a mixture, admixture, or solution in a second vessel (for example, at a second location) containing other ingredients of the oral care composition or personal care composition.

As used herein, “stilbenoid” or “stilbenoids” refers to a group of naturally occurring phenolic compounds found in various plant species. Generally, stilbenoids share a common backbone structure known as stilbene. However, the specific compounds may differ in the placement and position of substituents. Stilbenoids are classified as phytoalexins. Resveratrol. is one type of stilbenoid. As used herein, unless otherwise specified, “resveratrol” is meant to refer a compound with the following structure:

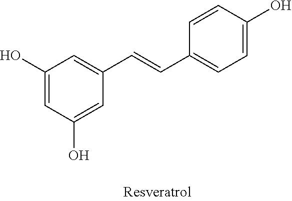 [SMILES: Oc1ccc(/C=C/c2cc(O)cc(O)c2)cc1]

In the present invention, it has been found that amine fluoride and stilbenoid, e.g., resveratrol, form a complex when they are mixed. It has been further found that the addition of stillbenoid, e.g., resveratrol, to a composition (e.g., toothpaste and mouthwash) containing an amine base, a fluoride ion source and a zinc source, increases the anti-inflammation ability of the composition in both fresh and aged formulations. Without intending to be bound to any theory, it is believed that the formation of amine fluoride-resveratrol complex may improve the stability of resveratrol in toothpaste and MW formulations.

In an aspect, the disclosure is directed to an oral care composition (Composition 1.0) comprising
an amine base,
a fluoride source,
a zinc source selected from zinc lactate and zinc citrate; and
one or more stilbenoid(s) (e.g., resveratrol).

For example, the present disclosure contemplates any of the following compositions (unless otherwise indicated, values are given as percentage of the overall weight of the composition):
1.1 Composition 1, wherein the amine base is a primary amine, secondary amine, tertiary amine or a combination thereof.
1.2 Any of the preceding compositions, wherein the amine base comprises or consists of a primary amine base.
1.3 Any of the preceding compositions, wherein the amine base comprises or consists of a secondary amine base.
1.4 Any of the preceding compositions, wherein the amine base comprises or consists of a tertiary amine base.
1.5 Any of the preceding compositions, wherein the amine base is plant-derived.
1.6 Any of the preceding compositions, wherein the amine base is animal-derived.
1.7 Any of the preceding compositions, wherein the amine base is derived from bovine tallow.
1.8 Any of the preceding compositions, wherein the amine base is derived from rapeseed oil or from rice bran oil.
1.9 Any of the preceding compositions, wherein the amine base is a linear or branched fatty amine or polyamine, or mixtures thereof
1.10 The preceding composition, wherein the amine base is a saturated or unsaturated C₁₂₋₂₀ alkyl amine base or a saturated or unsaturated C₁₂₋₂₀ alkyl polyamine base, or mixtures thereof.
1.11 Any of the preceding compositions, wherein the amine base is a myristyl, palmityl, linoleyl, oleyl, or stearyl amine or polyamine, or combinations thereof.
1.12 Any of the preceding compositions, wherein the amine base is a polyamine (e.g., a monoamine base, a diamine base and/or a triamine base).
1.13 Any of the preceding compositions, wherein the amine base is a monoamine base.
1.14 Any of the preceding compositions, wherein the amine base is a diamine base.
1.15 Any of the preceding compositions, wherein the amine base is a triamine base.
1.16 Any of the preceding compositions, wherein the amine base comprises one or more of N′-octadecyltrimethylendiamine-N,N,N′-tris(2-ethanol), and/or oleyldiamine ethoxylate and/or N-octadeca-9-enylamine, and/or alkyl trihydroxyethyl propylenediamine.
1.17 Any of the preceding compositions, wherein the amine base is N′-octadecyltrimethylendiamine-N,N,N′-tris(2-ethanol).
1.18 Any of the preceding compositions, wherein the amine base is N-octadeca-9-enylamine.
1.19 Any of the preceding compositions, wherein the amine base and fluoride ion source form amine fluoride in situ.
1.20 Any of the preceding compositions, further comprising an acid.
1.21 The preceding composition, wherein the acid is an organic acid (e.g., lactic acid, citric acid, tartaric acid, fumaric acid, malic acid), phosphoric acid or hydrochloric acid.
1.22 The preceding composition, wherein the organic acid is an aliphatic di- or tri-carboxylic acid in free or salt form.
1.23 Any of the preceding compositions, further comprising malic acid.
1.24 Any of the preceding compositions, further comprising hydrochloric acid.
1.25 Any of the preceding compositions, further comprising phosphoric acid.
1.26 Any of the preceding compositions, wherein the acid is not hydrofluoric acid.
1.27 Any of the preceding compositions, wherein the composition is substantially free of hydrofluoric acid (e.g., less than 0.001 wt. % hydrofluoric acid).
1.28 Any of the preceding compositions, wherein the amine base, fluoride ion source, and the acid form amine fluoride in situ.
1.29 Any of the preceding compositions, wherein the fluoride source is selected from one or more of sodium fluoride, potassium fluoride, sodium fluorosilicate, ammonium fluorosilicate, amine fluoride, ammonium fluoride, and combinations thereof.
1.30 Any of the preceding compositions, wherein the fluoride is sodium fluoride.
1.31 Any of the preceding compositions, wherein the composition comprises less than 0.01 wt. % stannous fluoride.
1.32 Any of the preceding compositions, wherein the composition comprises less than 0.001 wt. % stannous fluoride.
1.33 Composition 1.19 or 1.28, wherein the amine fluoride formed is one or more of N′-octadecyltrimethylendiamine-N,N,N′-tris(2-ethanol)-dihydrofluoride (olaflur) or N-octadeca-9-enylamine hydrofluoride (dectaflur).
1.34 Composition 1.19 or 1.28, wherein the amine fluoride formed is N′-octadecyltrimethylendiamine-N,N,N′-tris(2-ethanol)-dihydrofluoride (olaflur).
1.35 Composition 1.19 or 1.28, wherein the amine fluoride formed is N-octadeca-9-enylamine hydrofluoride (dectaflur).
1.36 Any of the preceding compositions, wherein the zinc source is zinc lactate.
1.37 Any of the preceding compositions, wherein the zinc source is zinc citrate.
1.38 Any of the preceding compositions, wherein the amine base is present in an amount of about 0.01 wt. % to about 5 wt. %, about 0.01 wt. % to about 3 wt. %, or about 0.1 wt. % to about 1 wt. % based on the total weight of the composition.
1.39 Any of the preceding compositions, wherein the amine base is present in an amount of about 0.5 wt. % to about 2.5 wt. %, about 1 wt. % to about 2 wt. %, about 1.2 wt. % to about 1.4 wt. %, or about 1.3 wt. %, based on the total weight of the composition.
1.40 Any of the preceding compositions, wherein the amine base is present in an amount of about 0.1 wt. % to about 0.5 wt. %, or about 0.15 wt. % to about 0.25 wt. %, based on the total weight of the composition.
1.41 The preceding composition wherein the fluoride ion source is present in an amount of 0.005wt. % to 2.5wt. % (e.g., about 0.025 wt. % to about 0.145 wt. %), about 0.1 wt. % to about 0.5 wt. %, or about 0.01 wt. % to about 0.03 wt. %, based on the total weight of the composition.
1.42 Any of the preceding compositions, wherein the total fluoride content of the composition is in an amount of from 50 to 25,000 ppm (e.g., 750 -7000 ppm, e.g., 1000-5500 ppm, e.g., about 250 ppm, 500 ppm, 1000 ppm, 1100 ppm, 1400 ppm, 1450 ppm, 2800 ppm, 5000 ppm, or 25000 ppm).
1.43 Any of the preceding compositions, wherein the zinc source is present in an amount of about 0.1 wt. % to about 2.5 wt. %, e.g., about 0.5 wt. % or about 2.0 wt. %, based on the total weight of the composition.
1.44 Any of the preceding compositions, wherein the zinc source is present in an amount of about 0.1 wt. % to about 0.2 wt. %, e.g., about 0.17 wt. % to about 0.18 wt. %, based on the total weight of the composition.
1.45 Any of the preceding compositions, wherein the zinc source is zinc lactate present in an amount of about 0.5 wt. %, based on the total weight of the composition.
1.46 Any of the preceding compositions, wherein the zinc source is zinc lactate present in an amount of about 0.1 wt. % to about 0.25 wt. %, or 0.2 wt. %, based on the total weight of the composition.
1.47 Any of the preceding compositions, wherein the zinc source is zinc citrate present in an amount of about 2.0 wt. %, based on the total weight of the composition.
1.48 Any of the preceding compositions, further comprising polyvinyl pyrrolidone in an amount of about 0.1 wt. % to about 1.00 wt. %, based on the total weight of the composition.
1.49 Any of the preceding compositions, comprising an acid (e.g., hydrochloric acid) in an amount of about 0.1 wt. % to about 1.0 wt. % (e.g., about 0.7 wt. % to about 0.9 wt. %), based on the total weight of the composition.
1.50 Any of the preceding compositions, comprising malic acid in an amount of about 0.03 wt. % to about 0.07 wt. %, based on the total weight of the composition.
1.51 Any of the preceding compositions, comprising a cellulose derivative (e.g., hydroxyethyl cellulose) in an amount of about 1 wt. % to about 2 wt. %, based on the total weight of the composition.
1.52 Any of the preceding compositions, further comprising an amino acid, (e.g., a basic amino acid) (e.g., arginine) present in an amount corresponding to 1% to 15%, e.g., 3 wt. % to 10 wt. % of the total composition weight, about e.g., 1.5%, 4%, 5%, or 8%, wherein the weight of the amino acid (e.g., basic amino acid) is calculated as free form.
1.53 Any of preceding compositions, wherein the composition is ethanol-free.
1.54 Any of the preceding compositions, wherein the pH is below 7, e.g., a pH of about 3-6, e.g., a pH of about 4-5.
1.55 Any of the preceding compositions, further comprising an effective amount of one or more alkali phosphate salts, e.g., sodium, potassium or calcium salts, e.g., selected from alkali dibasic phosphate and alkali pyrophosphate salts, e.g., alkali phosphate salts selected from sodium phosphate dibasic, potassium phosphate dibasic, dicalcium phosphate dihydrate, calcium pyrophosphate, tetrasodium pyrophosphate, tetrapotassium pyrophosphate, sodium tripolyphosphate, disodium hydrogenorthophoshpate, monosodium phosphate, pentapotassium triphosphate and mixtures of any of two or more of these, e.g., in an amount of 0.01-20%, e.g., 0.1-8%, e.g., e.g., 0.1 to 5%, e.g., 0.3 to 2%, e.g., 0.3 to 1%, e.g., about 0.01%, about 0.1%, about 0.5%, about 1%, about 2%, about 5%, about 6%, by weight of the composition.
1.56 The preceding composition, wherein the polyphosphate is tetrasodium pyrophosphate.
1.57 The preceding composition, wherein the tetrasodium pyrophosphate is from 0.1-1.0 wt. % (e.g., about 0.5 wt. %).
1.58 Any of the preceding compositions, further comprising an abrasive or particulate (e.g., silica).
1.59 Any of the preceding compositions, further comprising a nonionic surfactant, wherein the nonionic surfactant is in an amount of from 0.5 -5%, e.g., 1-2%, selected from poloxamers (e.g., poloxamer 407), polysorbates (e.g., polysorbate 20), polyoxyl hydrogenated castor oil (e.g., polyoxyl 40 hydrogenated castor oil), polyglyceryl 4-caprate, and mixtures thereof.
1.60 The preceding composition, wherein the poloxamer nonionic surfactant has a polyoxypropylene molecular mass of from 3000 to 5000 g/mol and a polyoxyethylene content of from 60 to 80 mol %, e.g., the poloxamer nonionic surfactant comprises poloxamer 407.
1.61 Any of the preceding compositions, further comprising a humectant selected from glycerin, sorbitol, xylitol, propylene glycol in an amount of about 10-70 wt. % based on the total weight of the composition.
1.62 Any of the preceding compositions, comprising a humectant selected from glycerin and sorbitol.
1.63 Any of the preceding compositions, further comprising a flavoring, fragrance and/or coloring agent.
1.64 Any of the preceding compositions, comprising one or more flavoring agents selected from saccharin and sucralose (e.g., saccharin in an amount of about 0.02 wt. % and sucralose in an amount of about 0.007 wt. % to about 0.01 wt. %).
1.65 The preceding composition, further comprising glycerin in an amount of about 2.0 wt. % to about 3.5 wt. %, based on the total weight of the composition.
1.66 Any of the preceding compositions, further comprising a thickening agent selected from the group consisting of carboxyvinyl polymers, hydroxyethyl cellulose and water-soluble salts of cellulose ethers (e.g., sodium carboxymethyl cellulose and sodium carboxymethyl hydroxyethyl cellulose).
1.67 Any of the preceding compositions, further comprising an antibacterial agent selected from halogenated diphenyl ether (e.g. triclosan), herbal extracts and essential oils (e.g., rosemary extract, tea extract, magnolia extract, thymol, menthol, eucalyptol, geraniol, carvacrol, citral, honokiol, catechol, methyl salicylate, epigallocatechin gallate, epigallocatechin, gallic acid, miswak extract, sea-buckthorn extract), bisguanide antiseptics (e.g., chlorhexidine, alexidine or octenidine), quaternary ammonium compounds (e.g., cetylpyridinium chloride (CPC), benzalkonium chloride, tetradecylpyridinium chloride (TPC), N-tetradecyl-4-ethylpyridinium chloride (TDEPC), phenolic antiseptics, hexetidine, octenidine, sanguinarine, povidone iodine, delmopinol, salifluor, sanguinarine, propolis and oxygenating agents (e.g., buffered sodium peroxyborate or peroxycarbonate), phthalic acid and its salts, monoperthalic acid and its salts and esters, ascorbyl stearate, oleoyl sarcosine, alkyl sulfate, dioctyl sulfosuccinate, salicylanilide, domiphen bromide, delmopinol, octapinol and other piperidino derivatives, nicin preparations, chlorite salts; and mixtures of any of the foregoing.
1.68 Any of the preceding compositions, further comprising an antioxidant, e.g., selected from the group consisting of Co-enzyme Q10, PQQ, Vitamin C, Vitamin E, Vitamin A, BHT, anethole-dithiothione, and mixtures thereof.
1.69 Any of the preceding compositions, further comprising a whitening agent selected from the group consisting of metal chlorites, perborates, percarbonates, peroxyacids, hypochlorites, and combinations thereof.
1.70 Any of the preceding compositions, further comprising an agent that interferes with or prevents bacterial attachment, e.g. ethyl lauroyl arginiate (ELA) or chitosan.
1.71 Any of the preceding compositions, wherein the oral composition may be any of the following oral compositions selected from the group consisting of: a toothpaste or a dentifrice, a mouthwash or a mouth rinse, a topical oral gel, sprays, powders, strips, chewing gum, ribbon, foam, mousse, floss and a denture cleanser.
1.72 Any of the preceding compositions, wherein the composition comprises quaternary ammonium surfactant (e.g., a pyridinium surfactant).
1.73 The composition of 1.72, wherein the quaternary ammonium surfactant comprises a pyridinium surfactant selected from the group consisting of: cetylpyridinium chloride, tetradecylpyridinium chloride, N-tetradecyl-4-ethyl pyridinium chloride, domiphen bromide, or mixtures thereof
1.74 The composition of 1.73, wherein the pyridinium surfactant is cetylpyridinium chloride (CPC).
1.75 Any of the preceding compositions, wherein the stilbenoid is selected from the group consisting of: resveratrol, pterostibene, gnetol, piceatannol, oxyresveratrol, trans-diptoinonesin B, hoeaphenol, piceid, 4-methoxy-(E)-resveratrol 3-O-rutinoside, rhaponticin, dihydro-resveratrol and combinations thereof
1.76 The composition of 1.75, wherein the stilbenoid (e.g., resveratrol) is present in the amount from 0.005%-0.5% by wt.
1.77 The oral care composition of 1.76, wherein the stilbenoid is resveratrol.
1.78 The oral care composition of 1.77, wherein the resveratrol is selected from:

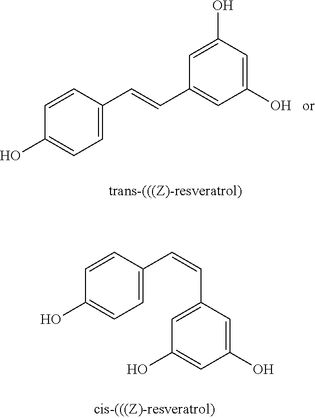 [SMILES: Oc1ccc(/C=C/c2cc(O)cc(O)c2)cc1 ; Oc1ccc(/C=C\c2cc(O)cc(O)c2)cc1]

1.79 Any of the preceding oral care compositions, wherein the stilbenoid (e.g., resveratrol) is derived from one or more plant extract(s).
1.80 The preceding oral care composition, wherein the stilbenoid is resveratrol derived from polygonum cuspidatum.
1.81 Any of the preceding oral care compositions, wherein the composition comprises a complex that forms from the combination of amine fluoride and resveratrol.
1.82 The composition of 1.81, wherein the complex comprises an amine bound to resveratrol (e.g., having a chemical structure: resveratrol—amine).
1.83 Any of the preceding wherein the composition comprises:
   oleyldiamine ethoxylate (e.g, oleyldiamine ethoxylate from rapeseed oil) (e.g., from 0.05%-1% by wt.);
   Zinc lactate (e.g., from 0.05%-2% by wt.) (e.g., about 0.2% by wt.); and
   Resveratrol (e.g., from 0.005%-0.5% by wt.) (e.g., about 0.01% by wt.) ((e.g., about 0.025% by wt.);
   Sodium fluoride (e.g., from 0.1%-1% by wt.); and
   Hydrochloric acid (e.g., from 0.1%-0.75% by wt.);

Wherein the composition further comprises amine fluoride that is formed in-situ using oleyldiamine ethoxylate as the amine base.
1.84 Any of the preceding wherein the composition comprises:
   Alkyl trihydroxyethyl propylenediamine; (e.g., from 0.05%-0.5% by wt.);
   Zinc lactate (e.g., from 0.05%-2% by wt.) (e.g., about 0.2% by wt.); and
   Resveratrol (e.g., from 0.005%-0.5% by wt.) (e.g., about 0.01% by wt.) ((e.g., about 0.025% by wt.);
   Sodium fluoride (e.g., from 0.1%-1% by wt.); and
   Hydrochloric acid (e.g., from 0.1%-0.75% by wt.);

Wherein the composition further comprises amine fluoride that is formed in-situ using alkyl trihydroxyethyl propylenediamine as the amine base.
1.85 Any of the preceding compositions wherein the composition comprises:
   Amine base (e.g., oleyldiamine ethoxylate) (e.g., oleyldiamine ethoxylate from rapeseed oil or alkyl trihydroxyethyl propylenediamine) (e.g., from 0.05%-1% by wt.);
   Zinc lactate (e.g., from 0.05%-2% by wt.) (e.g., about 0.2% by wt.); and
   Resveratrol (e.g., from 0.005%-0.5% by wt.) (e.g., about 0.01% by wt.) ((e.g., about 0.025% by wt.).
1.86 Any of the preceding compositions, wherein the composition is a mouthwash.
1.87 Any of the preceding compositions, wherein the oral care composition is free of stannous fluoride.
1.88 Any of the preceding compositions wherein the composition comprises:
   Amine base (e.g., oleyldiamine ethoxylate) (e.g., oleyldiamine ethoxylate from rapeseed oil or alkyl trihydroxyethyl propylenediamine) (e.g., from 0.05%-1% by wt.);
   Zinc lactate (e.g., from 0.05%-2% by wt.) (e.g., about 0.2% by wt.);
   Sodium fluoride (e.g., from 0.01%-1% by wt.); and
   Resveratrol (e.g., from 0.005%-0.5% by wt.) (e.g., about 0.01% by wt.) ((e.g., about 0.025% by wt.).
1.89 The oral care composition of 1.88, wherein the composition is a mouthwash.
1.90 Any of the preceding compositions, wherein the oral care composition comprises:
   Amine base (e.g., oleyldiamine ethoxylate) (e.g., oleyldiamine ethoxylate from rapeseed oil or alkyl trihydroxyethyl propylenediamine) (e.g., from 0.05%-1% by wt.);
   Zinc lactate (e.g., from 0.05%-2% by wt.) (e.g., about 0.2% by wt.); and
   Resveratrol (e.g., from 0.005%-0.5% by wt.) (e.g., about 0.01% by wt.) ((e.g., about 0.025% by wt.);
   Xylitol (e.g., from 0.5%-7.5% by wt.);
   Polyvinylpyrrolidone (e.g., from 0.05%-1% by wt.)
1.91 Any of the preceding compositions wherein the composition comprises:
   Amine base (e.g., oleyldiamine ethoxylate) (e.g., oleyldiamine ethoxylate from rapeseed oil or alkyl trihydroxyethyl propylenediamine) (e.g., from 0.05%-1% by wt.);
   Zinc lactate (e.g., from 0.05%-2% by wt.) (e.g., about 0.2% by wt.); and
   Resveratrol (e.g., from 0.005%-0.5% by wt.) (e.g., about 0.01% by wt.) ((e.g., about 0.025% by wt.).

wherein the total fluoride content of the composition is in an amount of from 50 to 5,000 ppm (e.g., about 250 ppm).
1.92 Any of the preceding compositions wherein the composition comprises:
   Amine base (e.g., oleyldiamine ethoxylate) (e.g., oleyldiamine ethoxylate from rapeseed oil or alkyl trihydroxyethyl propylenediamine) (e.g., from 0.05%-1% by wt.);
   Zinc lactate (e.g., from 0.05%-2% by wt.) (e.g., about 0.2% by wt.);
   Sodium fluoride (e.g., from 0.01%-1% by wt.); and
   Resveratrol (e.g., from 0.005%-0.5% by wt.) (e.g., about 0.01% by wt.) ((e.g., about 0.025% by wt.).

wherein the total fluoride content of the composition is in an amount of from 50 to 5,000 ppm (e.g., about 250 ppm).
1.93 Any of the preceding compositions wherein the composition comprises:
   Amine base (e.g., oleyldiamine ethoxylate) (e.g., oleyldiamine ethoxylate from rapeseed oil or alkyl trihydroxyethyl propylenediamine) (e.g., from 0.05%-1% by wt.);
   Zinc lactate (e.g., from 0.05%-2% by wt.) (e.g., about 0.2% by wt.);
   Sodium fluoride (e.g., from 0.01%-1% by wt.); and
   Resveratrol (e.g., from 0.005%-0.5% by wt.) (e.g., about 0.01% by wt.) ((e.g., about 0.025% by wt.).

wherein the composition further contains amine fluoride formed in situ, and wherein the resveratrol also forms an amine-resveratrol complex.
1.94 Any of the preceding compositions wherein the composition comprises:
   Amine fluoride (e.g., from 0.05%-1% by wt.) (e.g., oleyldiamine ethoxylate) (e.g., oleyldiamine ethoxylate from rapeseed oil or alkyl trihydroxyethyl propylenediamine);
   Zinc lactate (e.g., from 0.05%-2% by wt.) (e.g., about 0.2% by wt.);
   Sodium fluoride (e.g., from 0.01%-1% by wt.); and
   Resveratrol (e.g., from 0.005%-0.5% by wt.) (e.g., about 0.01% by wt.) ((e.g., about 0.025% by wt.).

Wherein the amine fluoride is formed in situ from an amine base (e.g., oleyldiamine ethoxylate) (e.g., oleyldiamine ethoxylate from rapeseed oil or alkyl trihydroxyethyl propylenediamine) and sodium fluoride.
1.95 The oral care composition of any of the preceding compositions, wherein the composition is a mouthwash or a toothpaste.
1.96 The oral care composition of any of the preceding compositions, wherein the composition comprises the following:
   Water (e.g., from 15%-35% by wt.)
   Glycerin (e.g., from 15%-30% by wt.)
   Sorbitol (e.g., from 10%-25% by wt.)
   Thickening silica (e.g., from 1%-5% by wt.)
   Amorphous silica (e.g., from 10%-25% by wt.) oleyldiamine ethoxylate from rapeseed oil or alkyl trihydroxyethyl propylenediamine, and wherein amine fluoride is formed in situ
   Sodium fluoride (e.g., from 0.1%-1% by wt.)
   Zinc lactate (e.g., from 0.1%-2% by wt.)
   Resveratrol (e.g., where the resveratrol is an extract from polygonum cusipdatum) (e.g., from 0.005%-0.1% by wt.)
   Cocamidopropyl betaine (e.g., from 0.2%-2% by wt.)
   Hydroxyethylcellulose (e.g., from 0.5%-5% by wt.)
   Flavor, sweetener and colorant (e.g., from 0.5%-5% by wt.)
1.97 Any of the preceding compositions, for use in the treatment of periodontitis and/or gingivitis.

In another embodiment, the present disclose encompasses a composition obtained or obtainable by combining the ingredients as set forth in any of the preceding compositions.

In another embodiment, the present disclosure encompasses a method to improve oral health comprising applying an effective amount of the oral composition of any of the embodiments set forth above to the oral cavity of a subject in need thereof, e.g., a method to
i. reduce or inhibit formation of dental caries,
ii. reduce, repair or inhibit early enamel lesions, e.g., as detected by quantitative light-induced fluorescence (QLF) or electrical caries measurement (ECM),
iii. reduce or inhibit demineralization and promote remineralization of the teeth,
iv. reduce hypersensitivity of the teeth,
v. reduce or inhibit gingivitis,
vi. promote healing of sores or cuts in the mouth,
vii. inhibit microbial biofilm formation in the oral cavity,
viii. raise and/or maintain plaque pH at levels of at least pH 5.5 following sugar challenge,
ix. reduce plaque accumulation,
x. treat dry mouth,
xi. enhance systemic health, including cardiovascular health, e.g., by reducing potential for systemic infection via the oral tissues,
xii. whiten teeth,
xiii. reduce erosion of the teeth,
xiv. immunize (or protect) the teeth against cariogenic bacteria and their effects, and/or
xv. clean the teeth and oral cavity.

### Fluoride Ion Source

The oral care compositions may further include one or more fluoride ion sources, e.g., soluble fluoride salts. A wide variety of fluoride ion-yielding materials can be employed as sources of soluble fluoride in the present compositions. Examples of suitable fluoride ion-yielding materials are found in U.S. Pat. No. 3,535,421, to Briner et al.; U.S. Pat. No. 4,885,155, to Parran, Jr. et al. and U.S. Pat. No. 3,678,154, to Widder et al., each of which are incorporated herein by reference. Representative fluoride ion sources used with the present disclosure (e.g., Composition 1.0 et seq.) include, but are not limited to, sodium fluoride, potassium fluoride, sodium fluorosilicate, ammonium fluorosilicate, amine fluoride, ammonium fluoride, and combinations thereof. In certain embodiments the fluoride ion source includes sodium fluoride. Where the formulation comprises calcium salts, the fluoride salts are preferably salts wherein the fluoride is covalently bound to another atom, e.g., as in sodium monofluorophosphate, rather than merely ionically bound, e.g., as in sodium fluoride.

### Surfactants

In another embodiment, cationic surfactants useful in the present disclosure can be broadly defined as derivatives of aliphatic quaternary ammonium compounds having one long alkyl chain containing 8 to 18 carbon atoms such as lauryl trimethylammonium chloride, cetyl pyridinium chloride, cetyl trimethylammonium bromide, di-isobutylphenoxyethyldimethylbenzylammonium chloride, coconut alkyltrimethylammonium nitrite, cetyl pyridinium fluoride, and mixtures thereof. Illustrative cationic surfactants are the quaternary ammonium fluorides described in U.S. Pat. No. 3,535,421, to Briner et al., herein incorporated by reference. Certain cationic surfactants can also act as germicides in the compositions.

Illustrative nonionic surfactants that can be used in the compositions of the disclosure, e.g., any of Composition 1.0, et seq., can be broadly defined as compounds produced by the condensation of alkylene oxide groups (hydrophilic in nature) with an organic hydrophobic compound which may be aliphatic or alkylaromatic in nature. Examples of suitable nonionic surfactants include, but are not limited to, the Pluronics, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides and mixtures of such materials. In a particular embodiment, the composition of the disclosure comprises a nonionic surfactant selected from poloxamers (e.g., poloxamer 407), polysorbates (e.g., polysorbate 20), polyoxyl hydrogenated castor oils (e.g., polyoxyl 40 hydrogenated castor oil), betaines (such as cocamidopropylbetaine), and mixtures thereof.

Illustrative amphoteric surfactants that can be used in the compositions of the disclosure, e.g., any of Composition 1.0, et seq., include betaines (such as cocamidopropylbetaine), derivatives of aliphatic secondary and tertiary amines in which the aliphatic radical can be a straight or branched chain and wherein one of the aliphatic substituents contains about 8-18 carbon atoms and one contains an anionic water-solubilizing group (such as carboxylate, sulfonate, sulfate, phosphate or phosphonate), and mixtures of such materials.

The surfactant or mixtures of compatible surfactants can be present in the compositions of the present disclosure in 0.1% to 5%, in another embodiment 0.3% to 3% and in another embodiment 0.5% to 2% by weight of the total composition.

### Flavoring Agents

The oral care compositions of the disclosure, e.g., any of Composition 1.0 et seq., may also include a flavoring agent. Flavoring agents which are used in the practice of the present disclosure include, but are not limited to, essential oils and various flavoring aldehydes, esters, alcohols, and similar materials, as well as sweeteners such as sodium saccharin. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. Certain embodiments employ the oils of peppermint and spearmint.

The flavoring agent is incorporated in the oral composition at a concentration of 0.01 to 1.7% by weight.

### Chelating and Anti-Calculus Agents

The oral care compositions of the disclosure, e.g., any of Composition 1.0 et seq, may also include one or more chelating agents able to complex calcium found in the cell walls of the bacteria. Binding of this calcium weakens the bacterial cell wall and augments bacterial lysis.

Another group of agents suitable for use as chelating or anti-calculus agents in the present disclosure are the soluble pyrophosphates. The pyrophosphate salts used in the present compositions can be any of the alkali metal pyrophosphate salts. In certain embodiments, salts include tetra alkali metal pyrophosphate, dialkali metal diacid pyrophosphate, trialkali metal monoacid pyrophosphate and mixtures thereof, wherein the alkali metals are sodium or potassium. The salts are useful in both their hydrated and unhydrated forms. An effective amount of pyrophosphate salt useful in the present composition is generally enough to provide least 0.1 wt. % pyrophosphate ions, e.g., 0.1 to 3 wt. 5, e.g., 0.1 to 2 wt. %, e.g., 0.1 to 1 wt. %, e.g., 0.2 to 0.5 wt. %. The pyrophosphates also contribute to preservation of the compositions by lowering water activity.

### Polymers

The oral care compositions of the disclosure, e.g., any of Composition 1.0 et seq, also optionally include one or more polymers, such as polyethylene glycols, polyvinyl methyl ether maleic acid copolymers, polysaccharides (e.g., cellulose derivatives, for example carboxymethyl cellulose). Acidic polymers, for example polyacrylate gels, may be provided in the form of their free acids or partially or fully neutralized water-soluble alkali metals (e.g., potassium and sodium) or ammonium salts. Certain embodiments include 1:4 to 4:1 copolymers of maleic anhydride or acid with another polymerizable ethylenically unsaturated monomer, for example, methyl vinyl ether (methoxyethylene) having a molecular weight (M.W.) of about 30,000 to about 1,000,000. These copolymers are available for example as Gantrez AN 139(M.W. 500,000), AN 1 19 (M.W. 250,000) and S-97 Pharmaceutical Grade (M.W. 70,000), of GAF Chemicals Corporation.

Other operative polymers include those such as the 1:1 copolymers of maleic anhydride with ethyl acrylate, hydroxyethyl methacrylate, N-vinyl-2-pyrollidone, or ethylene, the latter being available for example as Monsanto EMA No. 1 103, M.W. 10,000 and EMA Grade 61, and 1:1 copolymers of acrylic acid with methyl or hydroxyethyl methacrylate, methyl or ethyl acrylate, isobutyl vinyl ether or N-vinyl-2-pyrrolidone.

Suitable generally, are polymerized olefinically or ethylenically unsaturated carboxylic acids containing an activated carbon-to-carbon olefinic double bond and at least one carboxyl group, that is, an acid containing an olefinic double bond which readily functions in polymerization because of its presence in the monomer molecule either in the alpha-beta position with respect to a carboxyl group or as part of a terminal methylene grouping. Illustrative of such acids are acrylic, methacrylic, ethacrylic, alpha-chloroacrylic, crotonic, beta-acryloxy propionic, sorbic, alpha-chlorsorbic, cinnamic, beta-styrylacrylic, muconic, itaconic, citraconic, mesaconic, glutaconic, aconitic, alpha-phenylacrylic, 2-benzyl acrylic, 2-cyclohexylacrylic, angelic, umbellic, fumaric, maleic acids and anhydrides. Other different olefinic monomers copolymerizable with such carboxylic monomers include vinylacetate, vinyl chloride, dimethyl maleate and the like. Copolymers contain sufficient carboxylic salt groups for water-solubility.

A further class of polymeric agents includes a composition containing homopolymers of substituted acrylamides and/or homopolymers of unsaturated sulfonic acids and salts thereof, in particular where polymers are based on unsaturated sulfonic acids selected from acrylamidoalykane sulfonic acids such as 2-acrylamide 2 methylpropane sulfonic acid having a molecular weight of about 1,000 to about 2,000,000, described in U.S. Pat. No. 4,842,847, Jun. 27, 1989 to Zahid, incorporated herein by reference.

Another useful class of polymeric agents includes polyamino acids, particularly those containing proportions of anionic surface-active amino acids such as aspartic acid, glutamic acid and phosphoserine, as disclosed in U.S. Pat. No. 4,866,161 Sikes et al., incorporated herein by reference.

In preparing oral care compositions, it is sometimes necessary to add some thickening material to provide a desirable consistency or to stabilize or enhance the performance of the formulation. In certain embodiments, the thickening agents are carboxyvinyl polymers, hydroxyethyl cellulose and water-soluble salts of cellulose ethers such as sodium carboxymethyl cellulose and sodium carboxymethyl hydroxyethyl cellulose. Natural gums such as karaya, gum arabic, and gum tragacanth can also be incorporated. Colloidal magnesium aluminum silicate or finely divided silica can be used as component of the thickening composition to further improve the composition's texture. In certain embodiments, thickening agents in an amount of about 0.5% to about 5.0% by weight of the total composition are used.

### Abrasives

In certain embodiments the disclosure, e.g., any of Composition 1.0 et seq, may comprise additional silica abrasives, sodium metaphosphate, potassium metaphosphate, aluminum silicate, calcined alumina, bentonite or other siliceous materials, or combinations thereof. Any silica suitable for oral care compositions may be used, such as precipitated silicas or silica gels. For example, synthetic amorphous silica. Silica may also be available as a thickening agent, e.g., particle silica. For example, the silica can also be small particle silica (e.g., Sorbosil AC43 from PQ Corporation, Warrington, United Kingdom).

### Water

Water is present in the oral compositions of the disclosure, e.g., any of Composition 1.0 et seq. Water, employed in the preparation of commercial oral compositions should be deionized and free of organic impurities. Water commonly makes up the balance of the compositions and includes 5% to 99%, e.g., 10%-20%, e.g., 25-35%, e.g., 40%-95%, e.g., 60%-95%, by weight of the oral compositions. This amount of water includes the free water which is added plus that amount which is introduced with other materials such as with sorbitol or silica or any components of the disclosure. The Karl Fischer method is a one measure of calculating free water.

### Humectants

Within certain embodiments of the oral compositions, it is also desirable to incorporate a humectant to reduce evaporation and also contribute towards preservation by lowering water activity. Certain humectants can also impart desirable sweetness or flavor to the compositions. The humectant, on a pure humectant basis, generally includes 1% to 70% in one embodiment or 30% to 65% in another embodiment by weight of the composition.

Suitable humectants include edible polyhydric alcohols such as glycerin, sorbitol, xylitol, propylene glycol as well as other polyols and mixtures of these humectants. Mixtures of glycerin and sorbitol may be used in certain embodiments as the humectant component of the compositions herein.

### pH Adjusting Agents

In some embodiments, the compositions of the present disclosure contain a buffering agent. Examples of buffering agents include anhydrous carbonates such as sodium carbonate, sesquicarbonates, bicarbonates such as sodium bicarbonate, silicates, bisulfates, phosphates (e.g., monopotassium phosphate, dipotassium phosphate, tribasic sodium phosphate, sodium tripolyphosphate, phosphoric acid), citrates (e.g. citric acid, trisodium citrate dehydrate), pyrophosphates (sodium and potassium salts) and combinations thereof. The amount of buffering agent is sufficient to provide a pH of about 3 to about 9, preferable about 4 to about 5, when the composition is dissolved in water, a mouth rinse base, or a toothpaste base. Typical amounts of buffering agent are about 5% to about 35%, in one embodiment about 10% to about 30%, in another embodiment about 15% to about 25%, by weight of the total composition.

The present disclosure in its method aspect involves applying to the oral cavity a safe and effective amount of the compositions described herein.

The compositions and methods according to the disclosure (e.g., Composition 1.0 et seq) can be incorporated into oral compositions for the care of the mouth and teeth such as toothpastes, transparent pastes, gels, mouthwashes, mouth rinses, sprays, foams, lozenges, mousses, toothpaste powders, tablets and chewing gum.

As used throughout, ranges are used as shorthand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls. It is understood that when formulations are described, they may be described in terms of their ingredients, as is common in the art, notwithstanding that these ingredients may react with one another in the actual formulation as it is made, stored and used, and such products are intended to be covered by the formulations described.

The following examples further describe and demonstrate illustrative embodiments within the scope of the present disclosure. The examples are given solely for illustration and are not to be construed as limitations of this disclosure as many variations are possible without departing from the spirit and scope thereof. Various modifications of the disclosure in addition to those shown and described herein should be apparent to those skilled in the art and are intended to fall within the appended claims.

### EXAMPLES

### Example 1

Toothpaste formulations having the formulas as indicated in Table 1 are prepared.

TABLE 1
Toothpaste formulations according to the
present disclosure (amounts in % by wt.)

| | Formulation 1 | | |
|---|---|---|---|
| | (negative control) | Formulation 2 | Formulation 3 |
| Surfactants | 5.5 | 5.5 | 5.5 |
| Amine base (e.g., | 1.4 | 1.4 | 1.4 |
| oleyldiamine | | | |
| ethoxylate from | | | |
| rapeseed oil) | | | |
| Hydrochloric acid | 0.69 | 0.69 | 0.69 |
| (32% solution) | | | |
| Sodium saccharin | 0.39 | 0.39 | 0.39 |
| Glycerin | 20 | 20 | 20 |
| Sorbitol (70% | 20 | 20 | 20 |
| solution) | | | |
| Amorphous silica | 19 | 19 | 19 |
| Thickening silica | 2.7 | 2.7 | 2.7 |
| Thickener | 1.7 | 1.7 | 1.7 |
| Zinc lactate | 0.5 | 0.5 | 0.5 |
| Resveratrol | — | 0.1 | 0.25 |
| Sodium fluoride | 0.31 | 0.31 | 0.31 |
| Fragrants and | 1.6 | 1.6 | 1.6 |
| colorants | | | |
| water | q.s. | q.s. | q.s. |

Formulations 1-3 contain 1.4% amine base, 0.31% sodium fluoride, and 0.5% zinc lactate. However, Formulation 1 is different from Formulations 2 and 3 in that formulation 1 (negative control) does not contain resveratrol, while Formulations 2 and 3 contain 0.1% or 0.25% resveratrol, respectively.

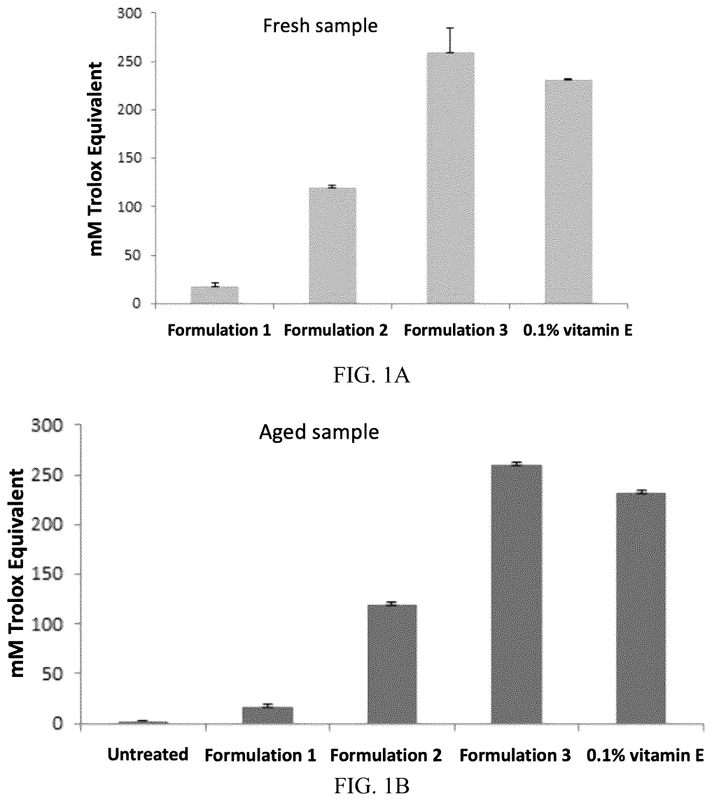

Formulations 1-3 as well as a formulation containing 0.1% vitamin E (positive control) are assessed for their antioxidation ability. Total Antioxidant Capacity Assay Kit (Abcam Catalog #: ab65329) is used to assess anti-oxidation capacity of the formulations. In this assay, Cu²⁺ is used as proxy for the ROS (Reactive Oxygen Species)/Oxidized form. The transfer of an electron from an antioxidant molecule converts Cu²⁺ (oxidized form) to Cu²⁺ (reduced form). Reduced Cu²⁺ ion chelates with a colorimetric probe, giving a broad absorbance peak at 570 nm, which is proportional to the total antioxidant capacity. The kit gives antioxidant capacity in Trolox equivalents. Trolox, a water-soluble vitamin E analog, serves as an antioxidant standard. The Assay is conducted by using Cu²⁺ working solution (made by diluting 1 part of the Cu²⁺ reagent in 49 parts Assay Buffer). 100 μl of each sample and standard are placed in a 96-well clear flat bottom plate. 100 μl of Cu²⁺ working solution is added to each well with samples or standards. After incubation, plate is measured for absorbance at 570 nm. Data analysis is performed by creating a linear standard curve by plotting the concentration and absorbance of the standards. Standard curve is used to determine the concentration of the samples. Both fresh and aged samples are assessed for their antioxidation ability. The aged samples are stored for 3 months at 40° C. and 75% relative humidity (RH). The results are shown in FIG. 1A (fresh samples) and FIG. 1B (aged samples). Formulations 2 and 3 show higher anti-oxidation activity compared to Formulation 1 in both fresh and aged samples. The anti-oxidation activity of Formulation 3 containing 0.25% resveratrol is higher than that of Formulation 2 containing 0.1% resveratrol. These results show that the addition of resveratrol to a toothpaste containing an amine base, a fluoride ion source and a zinc source increases the anti-oxidation ability of the composition in both fresh and aged samples.

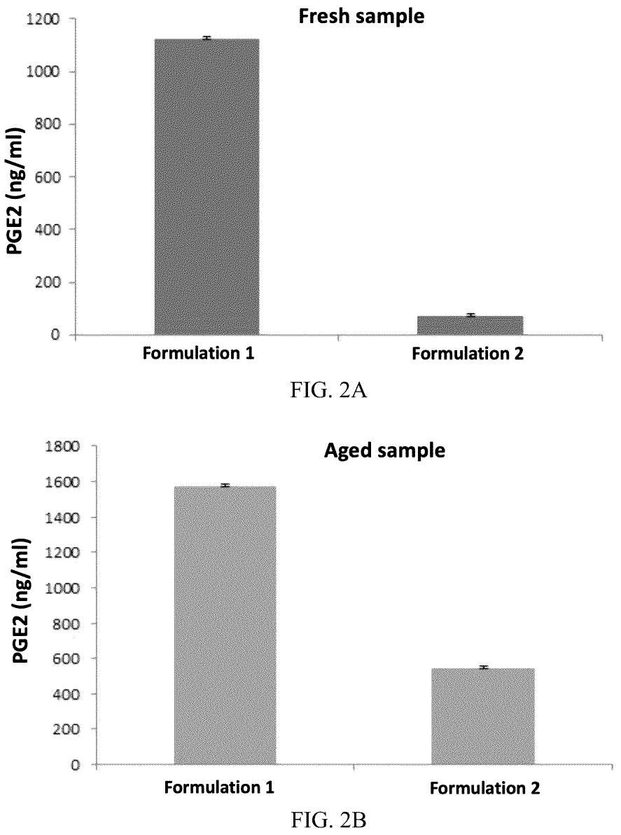

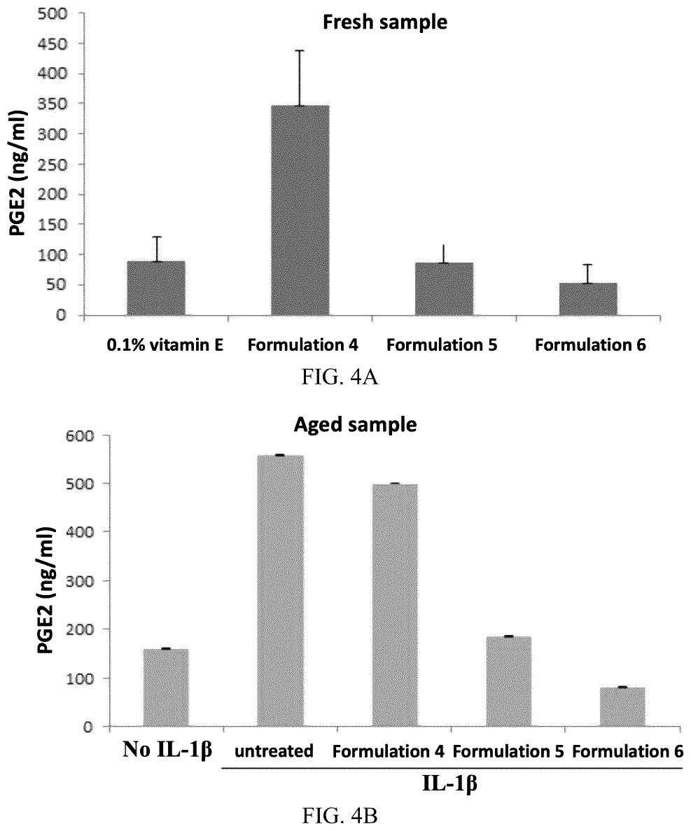

Next, Formulations 1 and 2 are assessed for their anti-inflammation activity. Cytokine PGE2 is used as an inflammation marker to evaluate the anti-inflammation efficacy of toothpaste formulas. Treatments are performed on human gingival tissue (Mattek Corporation, Ashland, MA) in the presence of IL-1β in the culture medium. Treating gingival tissue with IL-1β induces the expression of Cytokine PGE2 (FIG. 4B). The reduction of PGE2 level indicates that inflammation is reduced. The results are shown in FIG. 2A (fresh samples) and FIG. 2B (aged samples). The results show that the addition of resveratrol to a toothpaste containing an amine base, a fluoride ion source and a zinc source increases the anti-inflammation ability of the composition in both fresh and aged samples.

### Example 2

Mouthwash formulations having the formulas as indicated in Table 2 are prepared.

TABLE 2
Mouthwash formulations according to the
present disclosure (amounts in % by wt.)

| | Formulation 4 | | |
|---|---|---|---|
| | (negative | | |
| | control) | Formulation 5 | Formulation 6 |
| Surfactants | 0.14 | 0.14 | 0.14 |
| Amine base (e.g., | 0.16 | 0.16 | 0.16 |
| Alkyl trihydroxyethyl | | | |
| propylenediamine) | | | |
| Xylitol | 2.5 | 2.5 | 2.5 |
| Sodium saccharin | 0.02 | 0.02 | 0.02 |
| Glycerin | 20 | 20 | 20 |
| 40% Hydrofluoric | 0.034 | 0.034 | 0.034 |
| acid | | | |
| Polyvinylpyrollidone | 0.8 | 0.8 | 0.8 |
| Zinc lactate | 0.2 | 0.2 | 0.2 |
| Resveratrol | — | 0.01 | 0.025 |
| Sodium fluoride | 0.275 | 0.275 | 0.275 |
| Flavor and colorants | 0.12 | 0.12 | 0.12 |
| water | q.s. | q.s. | q.s. |

Formulations 4-6 contains 0.16% amine base, 0.275% sodium fluoride, and 0.2% zinc lactate. However, Formulation 4 is different from formulations 5 and 6 in that Formulation 4 (negative control) does not contain resveratrol, while Formulations 5 and 6 contain 0.01% or 0.025% resveratrol, respectively.

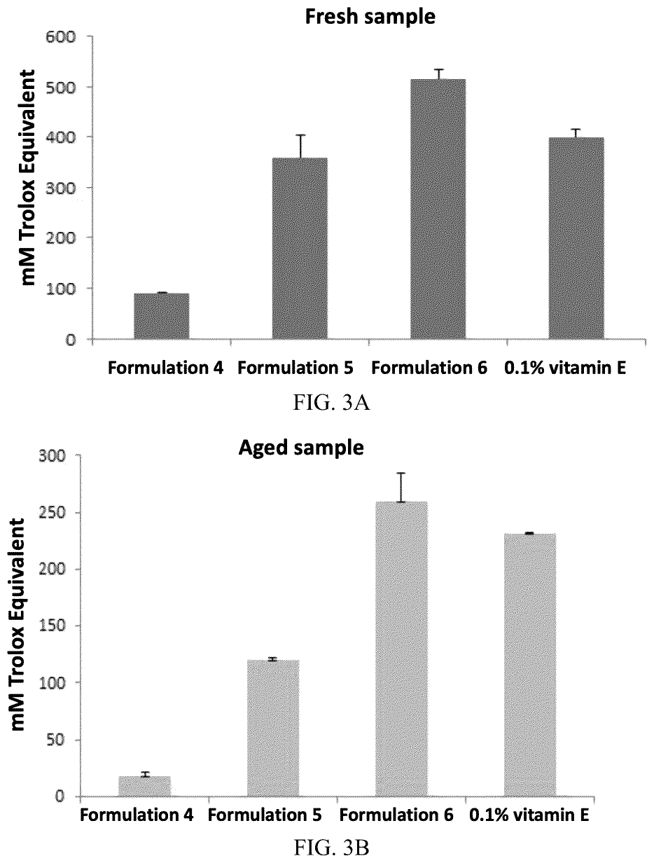

Formulations 4-6 as well as a formulation containing 0.1% vitamin E (positive control) are assessed for their antioxidation ability. Total Antioxidant Capacity Assay Kit (Abcam Catalog #: ab65329) is used to assess anti-oxidation capacity of the formulations. In this assay, Cu²⁺ is used as proxy for the ROS (Reactive Oxygen Species)/Oxidized form. The transfer of an electron from an antioxidant molecule converts Cu²⁺ (oxidized form) to Cu²⁺ (reduced form). Reduced Cu²⁺ ion chelates with a colorimetric probe, giving a broad absorbance peak at 570 nm, which is proportional to the total antioxidant capacity. The kit gives antioxidant capacity in Trolox equivalents. Trolox, a water-soluble vitamin E analog, serves as an antioxidant standard. The Assay is conducted by using Cu²⁺ working solution (made by diluting 1 part of the Cu²⁺ reagent in 49 parts Assay Buffer). 100 μl of each sample and standard are placed in a 96-well clear flat bottom plate. 100 μl of Cu²⁺ working solution is added to each well with samples or standards. After incubation, plate is measured for absorbance at 570 nm. Data analysis is performed by creating a linear standard curve by plotting the concentration and absorbance of the standards. Standard curve is used to determine the concentration of the samples. Both fresh and aged samples are assessed for their antioxidation ability. The aged samples are stored for 3 months at 40° C. and 75% relative humidity (RH). The results are shown in FIG. 3A (fresh samples) and FIG. 3B (aged samples). Formulations 5 and 6 show higher anti-oxidation activity compared to Formulation 4 in both fresh and aged samples. The anti-oxidation activity of Formulation 6 containing 0.025% resveratrol is higher than that of Formulation 5 containing 0.01% resveratrol. These results show that the addition of resveratrol to a mouthwash containing an amine base, a fluoride ion source and a zinc source increases the anti-oxidation ability of the composition in both fresh and aged samples.

Next, Formulations 4-6 as well as a formulation containing 0.1% vitamin E (positive control) are assessed for their anti-inflammation activity. Cytokine PGE2 is used as an inflammation marker to evaluate the anti-inflammation efficacy of mouthwash formulas. Treatments are performed on human gingival tissue (Mattek Corporation, Ashland, MA) in the presence of IL-1β in the culture medium. Treating gingival tissue with IL-1β induces the expression of Cytokine PGE2 (FIG. 4B). The reduction of PGE2 level indicates that inflammation is reduced. The results are shown in FIG. 4A (fresh samples) and FIG. 4B (aged samples). Formulations 5 and 6 show higher anti-inflammation activity compared to Formulation 4 in both fresh and aged samples. The anti-inflammation activity of Formulation 6 containing 0.025% resveratrol is higher than that of Formulation 5 containing 0.01% resveratrol. These results show that the addition of resveratrol to a toothpaste containing an amine base, a fluoride ion source and a zinc source increases the anti-inflammation ability of the composition in both fresh and aged samples.

### Example 3

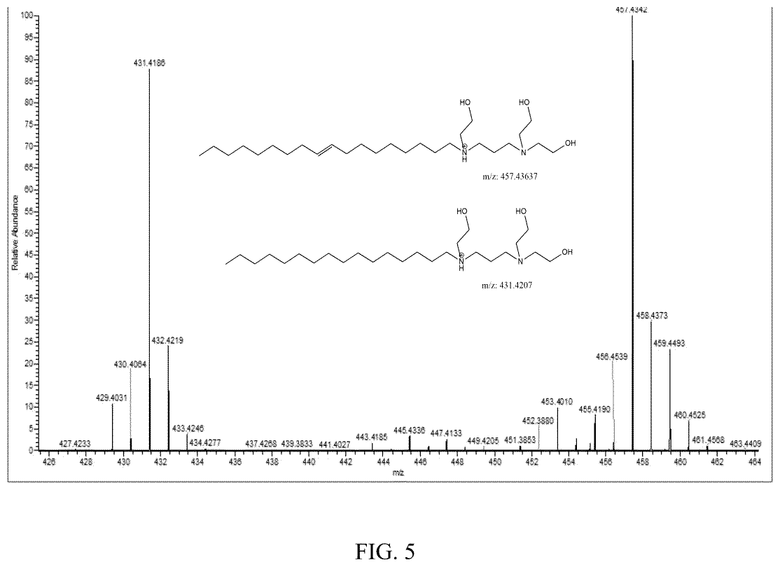

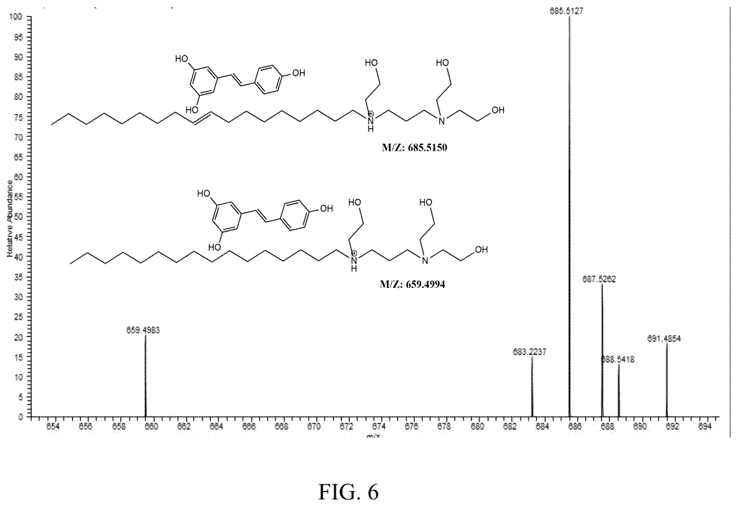

Samples are prepared with 10 mL 1000 ppm stock solution of amine fluoride and resveratrol with 50% methanol, respectively. A 1:1 ratio mixed 2 mL stock solution of amine fluoride and resveratrol is mixed by vortex. The high-pressure liquid chromatography-heated electrospray ionization-high resolution mass spectrometry (HPLC—HESI—HRMS) analysis is performed using a Q-Exactive™ Orbitrap™ mass spectrometry equipped with a HESI-II interface and Vanquish HPLC Systems from Thermo Fisher Scientific. The mobile phase is composited with 50% 10 mM AF-in pure water and 50% methanol. Samples are analyzed with a direct injection method in full scan MS mode from 100 to 100 m/z under positive polarity with electrospray ionization. And the resolution of 70,000 FWHM with 2.0×106 of Automatic Gain Control (AGC) target and 100 ms of maximum ion injection time are fixed during the analysis. The analyses are performed without a lock mass. The optimized parameter settings are: sheath, auxiliary and curtain gas flow rates at 35, 10 and 8 respectively, spray voltage 3.75 kV, capillary temperature 320° C., S-lens RF level 50, auxiliary gas heater temperature 400° C. Software used for operating the HILIC-HRMS was Xcalibur™0 (version 4.1). The liquid chromatograph-mass spectrometry (LC/MS) of amine ingredients from amine fluoride materials is shown in FIG. 5. Two peaks corresponding to amine ingredients are shown at 431.4186 and 457.4342. The liquid chromatograph-mass spectrometry (LC/MS) of the sample prepared by amine fluoride and resveratrol as disclosed above is shown in FIG. 6. Two peaks corresponding to amine-resveratrol complexes are shown at 659.4983 and 685.5127. Although complexes containing fluoride is not seen in LC/MS spectrometry, fluoride can be paired with amine, because the interaction between amine and fluoride does not interfere with the formation of amine-resveratrol complex. Thus, The LC/MS data demonstrates that amine fluoride and resveratrol form a complex. Without intending to be bound to any theory, the formation of this complex may potentially provide stability of resveratrol in toothpaste and MW formulations.

While the present disclosure has been described with reference to embodiments, it will be understood by those skilled in the art that various modifications and variations may be made therein without departing from the scope of the present disclosure as defined by the appended claims.

1. An oral care composition comprising
a) an amine base,
b) a fluoride source,
c) a zinc source selected from zinc lactate and zinc citrate; and
d) one or more stilbenoid(s).

2. The composition according to claim 1, wherein the amine base is a linear or branched fatty amine or polyamine.

3. The composition according to claim 1, wherein the amine base is a saturated or unsaturated C₁₂₋₂₀ alkyl amine base or a saturated or unsaturated C₁₂₋₂₀ alkyl polyamine base.

4. The composition according to claim 1, wherein the amine base is a myristyl, palmityl, linoleyl, oleyl, or stearyl amine or polyamine, or N′-octadecyltrimethylendiamine-N,N,N′-tris(2-ethanol), or N-octadeca-9-enylamine, and combinations thereof.

5. The composition according to claim 1, wherein the amine base is a polyamine (e.g., a monoamine base, a diamine base or a triamine base).

6. The composition according to claim 1, wherein the amine base is present in an amount of about 0.01 wt. % to about 5 wt. %, about 0.01 wt. % to about 3 wt. %, or about 0.1 wt. % to about 1 wt. % based on the total weight of the composition.

7. The composition according to claim 1, wherein the fluoride source is selected from one or more of sodium fluoride, potassium fluoride, ammonium fluoride, and combinations thereof.

8. The composition according to claim 1, wherein the fluoride source is present in an amount of 0.005 wt. % to 2.5 wt. %, about 0.1 wt. % to about 0.5 wt. %, or about 0.01 wt. % to about 0.03 wt. %, based on the total weight of the composition.

9. The composition according to claim 1, wherein the zinc source is zinc lactate.

10. The composition according to claim 1, wherein the zinc source is present in an amount of about 0.1 wt. % to about 2.5 wt. %, e.g., about 0.5 wt. % or about 2.0 wt. %, based on the total weight of the composition.

11. The composition according to claim 1, further comprising an acid selected from an organic acid, phosphoric acid or hydrochloric acid.

12. The composition according to claim 1, wherein the amine base, fluoride ion source, and the acid form amine fluoride in situ.

13. The oral care composition of claim 1, wherein the stilbenoid is present in the amount from 0.005%-0.5% by wt.

14. The oral care composition according to claim 1, wherein the stilbenoid is selected from the group consisting of:
resveratrol, pterostibene, gnetol, piceatannol, oxyresveratrol, trans-diptoinonesin B, hoeaphenol, piceid, 4-methoxy-(E)-resveratrol 3-O-rutinoside, rhaponticin, dihydro-resveratrol, and combinations thereof, optionally, wherein the stilbenoid is derived from one or more plant extract(s).

15. The oral care composition of claim 14, wherein the stilbenoid is resveratrol, optionally, wherein the resveratrol is derived from polygonum cuspidatum.

16. The oral care composition of claim 15, wherein the resveratrol is selected from:

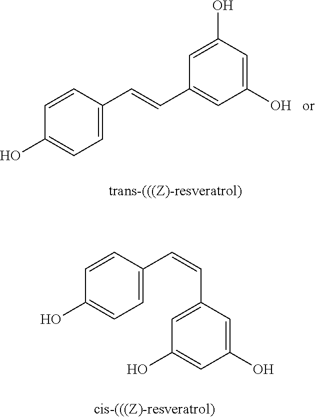 [SMILES: Oc1ccc(/C=C/c2cc(O)cc(O)c2)cc1 ; Oc1ccc(/C=C\c2cc(O)cc(O)c2)cc1]

17. (canceled)

18. (canceled)

19. The oral care composition of claim 1, wherein the composition comprises a complex that forms from the combination of amine fluoride and resveratrol.

20. The oral care composition of claim 19, wherein the complex comprises an amine bound to resveratrol.

21. The oral care composition of claim 1, wherein the composition comprises:
oleyldiamine ethoxylate;
Zinc lactate;
Resveratrol;
Sodium fluoride; and
Hydrochloric acid;
wherein the composition further comprises amine fluoride that is formed in-situ using oleyldiamine ethoxylate as the amine base.

22. The oral care composition of claim 1, wherein the composition comprises:
Alkyl trihydroxyethyl propylenediamine;
Zinc lactate;
Resveratrol;
Sodium fluoride; and
Hydrochloric acid;
wherein the composition further comprises amine fluoride that is formed in-situ using alkyl trihydroxyethyl propylenediamine as the amine base.
